(19) United States
(12) Patent Application Publication
(10) Pub. No.: US 2018/0353210 A1
(43) Pub. Date: Dec. 13, 2018

(54) Device for Manually Removing Calluses

(71) Applicant: Tadeusz Wladyslaw Lipowicz, Toronto (CA)

(72) Inventor: Tadeusz Wladyslaw Lipowicz, Toronto (CA)

(21) Appl. No.: 15/618,299

(22) Filed: Jun. 9, 2017

(51) Int. Cl. A61B 17/54

(52) U.S. Cl. CPC A61B 17/54

(57) ABSTRACT

A hand tool for removing callused skin is disclosed. The tool includes a head portion having an abrasive surface configured to abrade the callused skin rubbing the abrasive surface on the hard skin. The elongated portion has opposite first and second ends, opposite first and second sides, and a length extending from the first end to the second end. The elongated member also has a first width which progressively narrows along the length from the first end to the second end. The elongated head portion additionally has a second width which is perpendicular to the first width, the second width progressively narrowing along the length from the second end to the first end. The device also includes a cylindrical handle portion which has opposite proximal and distal ends extending from the second end, the proximal end being adjacent the second end of the elongated head portion.

### BACKGROUND

The present disclosure relates to the removal of hard skin (callus) from the heel and the bottom and the sides of the foot. Five percent or over 16,000,000 of the US population experience callus or corn problems (Illinois Podiatric Medical Association). Then there is a portion of the population that does not consider callus to be a problem but rather a personal care item, not unlike toe nail clipping and then there is a significant percentage of people that pay attention to the appearance of the feet for esthetic reasons.

For most people the treatment consists of regular removal of callus, either softened with water, gel or cream or dry, using electric or manual tools. The manual tools most popular on the market today are rasps, files, scrapers and shavers, made of stainless steel or abrasives: from aluminum oxide to diamonds. The majority of the callus affected population handles the problem by regularly scrubbing, scraping or shaving the hardened skin off the feet using one of the above mentioned tools. The process requires considerable effort and time to expose a healthy skin under the callus.

The author of the invention, affected by a serious callus problem, has tried a variety of tools, electric and manual, over a period of 15 years. Over this time, the inventor has discovered that the various manual tools for callus removal previously on the market were difficult to use and often ineffective. Electrically powered callus removal tools have been shown to be particularly difficult to use as they require the user to either bend over or bend the leg closer to the body to allow the tool access to the foot. An improved callus removal tool which is easy to use was developed in response to the failings of the prior art tools.

### SUMMARY OF THE INVENTION

The present invention consists of a hand held device for abrading hard skin on a body part. The device includes an elongated head portion having an outer surface, opposite first and second ends, opposite first and second sides, a long axis, and a length extending along the long axis from the first end to the second end. The elongated head portion has first and second transverse cross-sectional diameters oriented perpendicular to the long axis, the first transverse diameter narrowing from the first end towards the second end, the second transverse diameter narrowing from the second end towards the first end. The elongated head portion has a concave surface formed on the first side and extending from the first end towards the second end with a convex surface formed on the second side and extending from the first end towards the second end. The outside surface of the elongated head is covered by an abrasive surface extending continuously between the first and second ends of the elongated head, the abrasive surface configured to abrade the hard skin on the body part when the abrasive surface is rubbed against the hard skin. The device also includes a cylindrical handle portion having opposite proximal and distal ends extending from the second end, the proximal end being adjacent the second end of the elongated head portion, the cylindrical handle portion being coaxially aligned with the head portion.

The present invention is also directed at a hand held device for abrading hard skin on a body part, the device consisting of an elongated head portion having an outer surface, opposite first and second ends, opposite first and second sides, a long axis, and a length extending along the long axis from the first end to the second end. The elongated head portion also has first and second transverse cross-sectional diameters oriented perpendicular to the long axis, the first transverse cross-sectional diameter tapering from a first widest portion at the first end towards a first narrowest portion at the second end, the second transverse cross-sectional diameter tapering from a second widest portion at the second end towards a second narrowest portion at the first end. The outside surface of the elongated head is covered by an abrasive surface extending continuously between the first and second ends of the elongated head, the abrasive surface configured to abrade the hard skin on the body part when the abrasive surface is rubbed against the hard skin. Finally, the device includes a cylindrical handle portion having opposite proximal and distal ends extending from the second end, the proximal end being adjacent the second end of the elongated head portion, the cylindrical handle portion being coaxially aligned with the head portion.

The invention is also directed at a hand held device for abrading hard skin on a body part. The device includes an elongated head portion having an outer abrasive surface configured to abrade the hard skin on the body part by rubbing the abrasive surface on the hard skin. The elongated portion has opposite first and second ends, opposite first and second sides, and a length extending from the first end to the second end. The elongated member also has a first width which progressively narrows along the length from the first end to the second end. The elongated head portion additionally has a second width which is perpendicular to the first width, the second width progressively narrowing along the length from the second end to the first end. The device also includes a cylindrical handle portion which has opposite proximal and distal ends extending from the second end, the proximal end being adjacent the second end of the elongated head portion.

### BRIEF DESCRIPTION OF THE DRAWINGS

FIG. 1 is an isometric view of a hand held device for removing hard skin made in accordance with the present invention showing the grip portion in its rearmost position;

FIG. 2 is a side view of the hand held device of FIG. 1 showing the grip portion in its foremost position;

FIG. 3 is a top view of the hand held device of FIG. 1 showing the grip portion in an intermediate position;

FIG. 4 is a bottom view of the hand held device of FIG. 1 showing the grip portion in its rearmost position;

FIG. 5 is a side view of the head portion of the device shown in FIG. 1;

FIG. 6 is a long sectional view taken along line A-A in FIG. 5;

FIG. 7 is a top view of the head portion shown in FIG. 5;

FIG. 8 is a cross sectional view taken along line B-B in FIG. 5;

FIG. 9 is a cross sectional view taken along line C-C in FIG. 5;

FIG. 10 is a cross sectional view taken along line D-D in FIG. 5;

FIG. 11 is a cross sectional view taken along line E-E in FIG. 5.

FIG. 12 is an expanded cross sectional view of detail F in FIG. 6 above.

### DETAILED DESCRIPTION

A callus removal tool should address two main issues relating to callus removal, namely the time and the effort required to accomplish the task. It should enable the user to do the job as effortlessly and as quickly as possible. Furthermore, the callus removal process requires a direct interaction between the skin of the foot and the working surface of the tool. For this reason, the properties of the working surface of the tool must be compatible with the properties of the surface of the foot. The tool must accommodate the curvatures of the foot, as well as the flexibility of the skin. To maximize ease of use, the hand-held, manual tool is applied to the surface of the foot using a back and forth or up and down, repeated motion. There are three main efforts involved in the process: (1) the effort to maintain the grip on the tool, (2) the effort to repeatedly move the tool forth and back at various, mostly close to vertical angles, and (3) the effort to maintain pressure between the working surface of the tool and the skin. The tool made in accordance with the present invention meets all these requirements and lessens the three main efforts required by any callus removal tool.

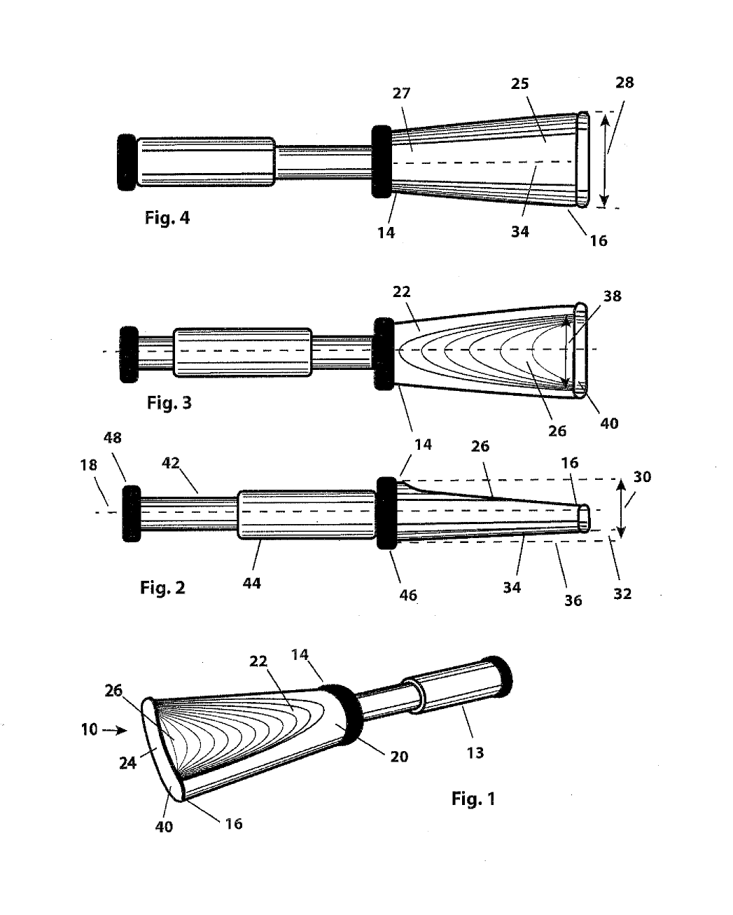

Referring firstly to FIGS. 1, 2, 3 and 4, a callus removal tool made in accordance with the present invention is shown generally as item 10 and consists of an elongated head portion 12 coupled to a handle portion 13. Head portion 12 has opposite ends 14 and 16, elongated axis 18, outer surface 20, opposite sides 22 and 24, concave trough 26 formed on side 22 and convex surface 25 formed on side 24 and end pad 40.

Elongated portion 12 has two cross-sectional diameters or widths. The first cross sectional diameter, shown as 28 in FIG. 4 gradually narrows (tapers) from end 16 towards end 14 such that width 28 is wider at end 16 than it is at end 14 by as much as one to two centimeters. Elongated portion 12 has a second cross-sectional diameter, shown as 30 in FIG. 2, which gradually narrows (tapers) from end 14 to end 16 such that cross-sectional diameter 30 is greater at end 14 then it is at end 16. Opposite concave trough 26 is a convex surface 25, the midpoint 34 of which extends between ends 14 and 16. Midpoint 34 gradually slopes towards axis 18 as the midpoint extends from end 14 to 16 such that the midpoint is sloped towards axis a distance 32 at end 14 relative to end 16 as shown by the orientation of midpoint 34 relative to line 36 in FIG. 2. Distance 32 can be as much as one centimeter.

As best seen in FIG. 3, concave trough 26 has a width 38 which tapers or narrows from end 16 at its widest, towards end 14 where it narrows down to a minimum. At its widest, width 38 is between 5 to 6 cm. The length of trough 26 between end 16 and end 14 is preferably in the range of 14 to 16 cm, but it will be appreciated that the length of the trough as well as the size of head portion 12 can be made either larger or smaller depending on how compact the device is to be made.

Handle portion 13 consists of a cylindrical member 42 over which a hollow tubular grip member 44 is mounted. Tubular grip member 44 is dimensioned such that the grip member can slide freely between a rear position as shown in FIG. 4 and a forward position shown in FIG. 2. Tubular grip member 44 preferably consists of a rubberized material which is sufficiently flexible and resilient to be deformed slightly when gripped firmly by the user. Tubular grip member 44 is dimensioned to have an internal diameter just slightly greater than the outside diameter of cylindrical member 42 so that when grasped firmly, grip member 44 deforms sufficiently to prevent the grip member from sliding on the cylindrical member. Hence, when grip 44 is in its forward position (FIG. 2) it will remain in that position as long as the user grasps the grip firmly. To slide grip 44 to its rear position (FIG. 4), the user simply relaxes his/her grip on member 44 and slides the member into position, and then grasps it firmly to keep it in position.

Tool 10 is provided with annular members 46 and 48 which are coaxially mounted to cylinder 42 by means known generally in the art. Members 46 and 48 each have a cross sectional diameter, with the diameter of annular member 46 being selected such that when tool 10 is placed on a flat surface such as a floor even the widest part of head portion 12 is slightly elevated above the floor. Preferably, the diameter of member 46 is greater than the diameter of member 48 so that when tool 10 is placed on the floor, head portion 12 is angled upward. If the diameters of members 46 and 48 are selected carefully, then member 48 may be made narrower than the maximum diameter of head portion 12 while still being able to elevate the head portion such that it does not touch the floor. Preferably member 46 is 5 to 6 cm in diameter, member 48 is 4 to 5 cm wide and head portion has a maximum width of 6 to 7 cm with the exact measurements of the relevant parts being selected such that the head portion of the tool can be rotated 360 degrees without touching the surface on which members 46 and 48 are rolled. Members 46 and 48 are preferably rubber coated so that when tool 10 is placed on the floor, the user can step on handle portion 13 to secure the tool in place, with the rubber coated annular members helping to prevent the tool from slipping. The user can change the orientation of the tool, and in particular, the orientation of head portion 12, simply by rolling the tool slightly until the desired orientation is achieved. Firmly placing the foot on the tool then holds the tool in the desired orientation.

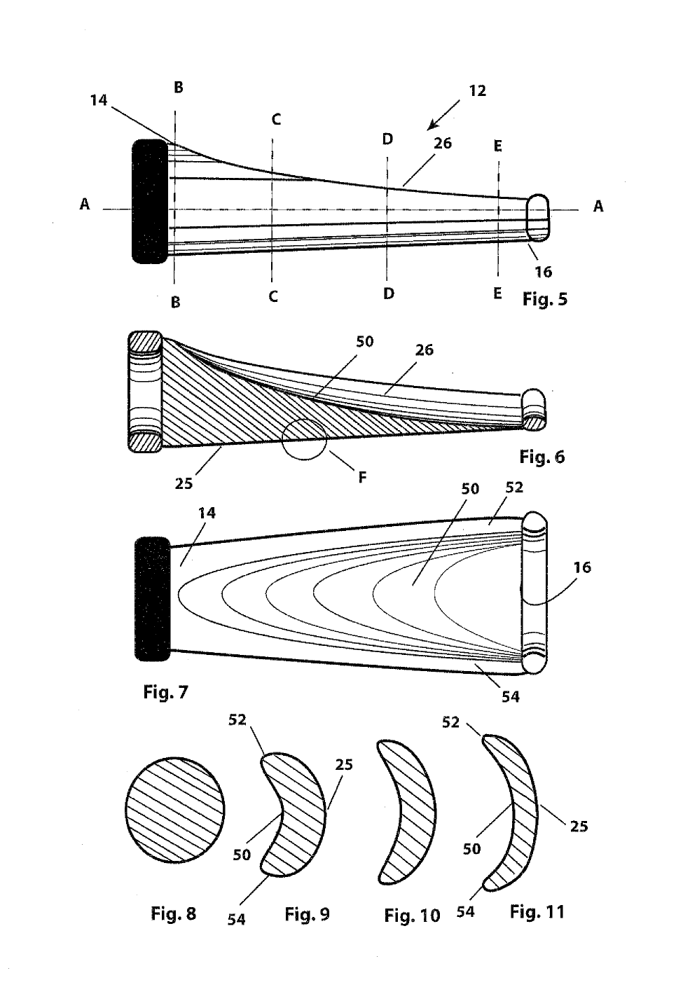

Referring now to FIGS. 5 through 11, trough 26 is concave and is conical in the sense that it is wider at end 16 than it is at end 14. Trough 26 has a bottom 50 which gradually curves between ends 14 and 16 and the bottom is surrounded at either side by ridges 52 and 54. The cross-sectional profile of head portion 12 changes from a circular profile adjacent end 14 as seen in FIG. 8 to a progressively more crescent shaped profile closer to end 16 as seen in FIGS. 9, 10 and 11. The crescent shaped profile is the result of the concave trough 26. The curved shape of bottom 50 and the curved shape of the walls of concave trough 26 make it easier to abrade callus skin located in curved portions of the body such as the heel of the foot. Since the width of trough 26 between ridges 52 and 54 changes from ends 16 to 14, it is possible to position the heel of the foot (or other body part) within the trough so as to maximize the efficient abrasion of callused skin on the heel or other body part. The convex side of the tool is designed to be compatible with the concave parts of the foot like the arch of the foot and the outer edge of the foot. Likewise, as best seen in FIGS. 9 to 11, ridges 52 and 54 are narrower closer to end 16 as compared to end 14, allowing for the user to abrade callus skin located in tight corners of a body part simply by rubbing the callus skin with either ridges 52 or 54 of the desired width.

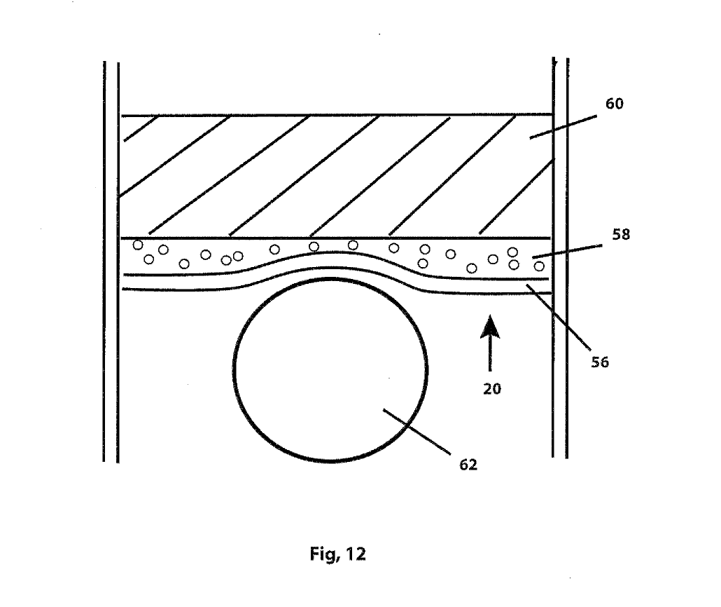

Referring now to FIG. 12, the head portion of the tool consists of a central core 60 with an outer surface 20, which is configured to be sufficiently abrasive to abrade callus skin by rubbing the outer surface against the skin. Outer surface 20 can be made abrasive by any number of methods known generally in the art, but preferably the abrasive surface is a resilient and flexible surface which is sufficiently flexible to conform to the shape of the body part 62 which the outer surface is pressed against. Most preferably, outer surface 20 consists of a coated abrasive flexible web 56 bonded to a resilient foam rubber pad 58 which is in turn mounted securely to core 60 of the head portion. The coated abrasive is essentially a flexible cloth having a granular abrasive bonded thereto by means known generally in the art. A variety of coated abrasives suitable for use with the present invention are commercially available, including emery cloth/paper coming in a variety of grit sizes (ratings). The coarser the grit size, the more abrasive outer surface 20 becomes. It is possible to produce the tool in a variety of different grit sizes ranging from finer to coarser. Flexible web 56 permits the abrasive surface to bend around body part 62 maximizing the surface area of contact between the body part and the abrasive surface. Resilient layer 58 provides a biasing force urging flexible web 56 towards the surface of body part 62, thereby increasing the abrasive action of surface 20.

The present invention has several advantages over prior callus removal tools. In particular, the invention addresses the problems related to the removal of the dry, dead skin from various callus affected areas of the foot, specifically:

Accessibility (providing a sufficient range of shapes and sizes, incorporated into the tool to access and work anywhere on the bottom and the edges of the foot);
Physical effort (maximizing the use of the easiest, least tiring motions in voluminous removal of the callus and providing an adjustable length of the handle);
Efficiency (increasing the quantity of the removed callus by increasing the friction and the area of contact between the abrasive and the surface of the foot), and
Time (increasing efficiency of the tool).

To achieve these benefits and efficiencies, the tool utilizes the following features. Firstly, a comfortable handle which can be repositioned for maintaining a sufficiently firm grip for the transfer of force from the hand to the working part of the tool. The grip portion of the invention provides for easy selection of the distance between the hand and the point of contact between the tool and the foot. The longer the distance the more force is required to maintain sufficient pressure at the contact with the foot, requiring a more comfortable grip.

Secondly, the head portion of the tool is designed such that the more tiring pressure applying effort, perpendicular to the surface of the skin, is minimized by the implementation of the upward slope in front of the forward moving tool, along either side of the tool and in front of back moving tool along either edge of the tool. The sharp edges of the abrasive slice off the callus during these motions. To reduce the number reciprocating motions and thus reduce the overall effort required to achieve a desired result, the shape of the head portion is designed such that each single reciprocal (up and down) motion of the tool head produces as much abrasion of callus as possible. This is accomplished by selecting the right abrasive mineral and grain size, applying enough pressure against the callus for the abrasive to slice off chunks of the skin with thickness approaching the vertically exposed grain height and providing an extended area of contact between the tool head and the foot. Since the tool head is wider at one end than at the other, a simple reciprocating up and down motion of the tool head causes a resulting back and forth biasing force to be applied to the callus as the tool head is moved up and down.

The resiliently deformable abrasive surface of the tool helps to maximize surface area of contact, and additionally increases the biasing force applied to the callused skin. The foam rubber pad underlying the abrasive coated web provides a changing-with-motion topography of the working surface of the tool. When pressed against the skin the created depression in the foam provides an enlarged contact area between the abrasive and the skin that follows the movement of the tool. There is a continuous, upward slope being formed in front of the moving tool, regardless of the direction of the movement. The slope contributes to the friction between the abrasive and the skin and consequently to the amount of skin being removed, The increased contact area between the tool and the skin, resulted from the formation of the depression in the foam-abrasive composite, also increases the amount of the removed skin, thus contributing to the heightened efficiency of the tool. The physical characteristics of the foam: thickness and degree of softness can be selected for the specific application of the tool to enable a more effective handling of specific types of callus problems. In addition, the resiliency of the foam rubber is selected to be as quick as possible to enable the abrasive to maintain contact with the skin behind the moving tool and to provide a ready slope in front of the tool when the direction of the motion is reversed.

The tool also has both concave and convex sides which are dimensioned to conform closely to the curvature of the foot, provide significantly larger areas of contact with the skin. This enables voluminous and uniform callus removal from the heel and the sides of the foot. The edges (items 52 and 54 of FIG. 7), due to their relatively small area of contact, can be used to efficiently remove local excess of callus overgrow.

In addition the tool can be used hands-free by placing it on the floor and holding it in place with one foot on the handle, while the other foot is worked on. All sides of the head portion of the tool can be used in this hands-free manner. To enable the stability and the maneuverability of the tool in the hands-free operation it is equipped with two rolling rings (annular members) that are covered with rubber.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art.

What is claimed is:

1. A hand held device for abrading hard skin on a body part, the device comprising:
a. An elongated head portion having an outer surface, opposite first and second ends, opposite first and second sides, a long axis, a length extending along the long axis from the first end to the second end, first and second transverse cross-sectional diameters oriented perpendicular to the long axis, the first transverse diameter narrowing from the first end towards the second end, the second transverse diameter narrowing from the second end towards the first end, a concave surface formed on the first side and extending from the first end towards the second end, a convex surface formed on the second side and extending from the first end towards the second end;
b. The outside surface of the elongated head being covered by an abrasive surface extending continuously between the first and second ends of the elongated head, the abrasive surface configured to abrade the hard skin on the body part when the abrasive surface is rubbed against the hard skin;
c. A cylindrical handle portion having opposite proximal and distal ends extending from the second end, the proximal end being adjacent the second end of the elongated head portion, the cylindrical handle portion being coaxially aligned with the head portion.

2. The device of claim 1 further comprising first and second annular rings coaxially projecting from the proximal and distal ends of the cylindrical handle, respectively, the first and second annular rings having first and second diameters, respectively, the first and second diameters being dimensioned such than when the device is placed on a flat surface the first and second annular rings position the elongated head portion so that the elongated head portion does not touch the flat surface.

3. The device of claim 2 wherein the second diameter is less than the first diameter.

4. The device of claim 2 further comprising a cylindrical hand grip formed on the cylindrical handle portion and positioned between the first and second annular rings, the cylindrical hand grip configured to slide freely between a first position adjacent the first annular ring and a second position adjacent the second annular ring, the hand grip being further configured such that grasping the hand grip tightly prevents the hand grip from sliding.

5. The device of claim 1 wherein the elongated head further comprises an inner core with the abrasive surface overlaying the inner core and wherein the abrasive surface comprises an outer abrasive layer overtop of a resilient pad, the outer abrasive layer comprising a coated abrasive flexible web and the resilient pad comprising a resilient flexible foam bonded to the coated abrasive flexible web, the resilient pad being mounted to the inner core such that the abrasive surface closely conforms to the outer surface of the head portion, the resilient pad having a thickness of at least 2 mm (Mr. Lipowicz, I am not certain what the minimum thickness of the pad is, but it seems to be at least 2 mm in the sample I have).

6. A hand held device for abrading hard skin on a body part, the device comprising:
a. An elongated head portion having an outer surface, opposite first and second ends, opposite first and second sides, a long axis, a length extending along the long axis from the first end to the second end, first and second transverse cross-sectional diameters oriented perpendicular to the long axis, the first transverse cross-sectional diameter tapering from a first widest portion at the first end towards a first narrowest portion at the second end, the second transverse cross-sectional diameter tapering from a second widest portion at the second end towards a second narrowest portion at the first end;
b. The outside surface of the elongated head being covered by an abrasive surface extending continuously between the first and second ends of the elongated head, the abrasive surface configured to abrade the hard skin on the body part when the abrasive surface is rubbed against the hard skin;
c. A cylindrical handle portion having opposite proximal and distal ends extending from the second end, the proximal end being adjacent the second end of the elongated head portion, the cylindrical handle portion being coaxially aligned with the head portion.

7. The device of claim 6 wherein the elongated head member has a cross-sectional profile which transitions smoothly from a circular cross-sectional profile at the second end of the elongated head portion to a crescent shaped cross-sectional profile at the first end of the elongated head portion so as to form a concave surface on the first side of the elongated head member which tapers from the crescent shaped cross-sectional profile of the first end towards the circular cross-sectional profile at the second end, the opposite second side being convex.

8. The device of claim 7 wherein the crescent shaped profile at the first end has a maximal width corresponding to the first widest portion of the first transverse cross-sectional diameter.

9. The device of claim 6 further comprising first and second annular rings coaxially projecting from the proximal and distal ends of the cylindrical handle, respectively, the first and second annular rings having first and second diameters, respectively, the first and second diameters being dimensioned such than when the device is placed on a flat surface the first and second annular rings position the elongated head portion so that the elongated head portion does not touch the flat surface.

10. The device of claim 9 wherein the second diameter is less than the first diameter.

11. The device of claim 8 further comprising first and second annular rings coaxially projecting from the proximal and distal ends of the cylindrical handle, respectively, the first and second annular rings having first and second diameters, respectively, the first and second diameters being dimensioned such than when the device is placed on a flat surface the first and second annular rings position the elongated head portion so that the elongated head portion does not touch the flat surface.

12. The device of claim 11 wherein the second diameter is less than the first diameter.

13. The device of claim 11 further comprising a cylindrical hand grip formed on the cylindrical handle portion and positioned between the first and second annular rings, the cylindrical hand grip configured to slide freely between a first position adjacent the first annular ring and a second position adjacent the second annular ring, the hand grip being further configured such that grasping the hand grip tightly prevents the hand grip from sliding.

14. The device of claim 8 wherein the elongated head further comprises an inner core with the abrasive surface overlaying the inner core and wherein the abrasive surface comprises an outer abrasive layer overtop of a resilient pad, the outer abrasive layer comprising a coated abrasive flexible web and the resilient pad comprising a resilient flexible foam bonded to the coated abrasive flexible web, the resilient pad being mounted to the inner core such that the abrasive surface closely conforms to the outer surface of the head portion, the resilient pad having a thickness of at least 2 mm.

15. A hand held device for abrading hard skin on a body part, the device comprising:
a. An elongated head portion having an outer abrasive surface configured to abrade the hard skin on the body part by rubbing the abrasive surface on the hard skin;
b. the elongated portion having opposite first and second ends, opposite first and second sides, a length extending from the first end to the second end, the elongated member having a first width which progressively narrows along the length from the first end to the second end, the elongated head portion having a second width perpendicular to the first width, the second width progressively narrowing along the length from the second end to the first end, and
c. A cylindrical handle portion having opposite proximal and distal ends extending from the second end, the proximal end being adjacent the second end of the elongated head portion.

16. The device of claim 15 wherein the first side of the elongated portion has a concave trough which has a widest point at the first end, the concave trough tapering gradually to a narrowest point adjacent the second end, the second side of the elongated portion being convex.

17. The device of claim 16 wherein the cylindrical handle portion is coaxially aligned with the head portion and further comprising first and second annular rings coaxially projecting from the proximal and distal ends of the cylindrical handle, respectively, the first and second annular rings having first and second diameters, respectively, the first and second diameters being dimensioned such than when the device is placed on a flat surface the first and second annular rings position the elongated head portion so that the elongated head portion does not touch the flat surface.

18. The device of claim 17 wherein the second diameter is less than the first diameter.

19. The device of claim 18 further comprising a cylindrical hand grip formed on the cylindrical handle portion and positioned between the first and second annular rings, the cylindrical hand grip configured to slide freely between a first position adjacent the first annular ring and a second position adjacent the second annular ring, the hand grip being further configured such that grasping the hand grip tightly prevents the hand grip from sliding.

20. The device of claim 19 wherein the elongated head further comprises an inner core with the outer abrasive surface overlaying the inner core and wherein the outer abrasive surface comprises a coated abrasive flexible web bonded to a resilient pad, the resilient pad being mounted to the inner core such that the abrasive surface closely conforms the elongated head portion, the resilient pad having a thickness of at least 2 mm.
